(19) United States
(12) Patent Application Publication
(10) Pub. No.: US 2008/0131420 A1
(43) Pub. Date: Jun. 5, 2008

(54) METHODS AND COMPOSITIONS FOR TREATING MUCOSAL INFLAMMATION

(75) Inventor: Francis E. O'Donnell, Town and Country, MO (US)

Correspondence Address: LAHIVE & COCKFIELD, LLP, ONE POST OFFICE SQUARE, BOSTON, MA, 02109-2127, US

(73) Assignee: Accentia, Inc., Tampa, FL (US)

(21) Appl. No.: 11/777,890

(22) Filed: Jul. 13, 2007

Related U.S. Application Data
(60) Provisional application No. 60/830,528, filed on Jul. 13, 2006.
(60) Provisional application No. 60/900,593, filed on Feb. 9, 2007.

(51) Int. Cl. A61K 38/47; A61K 31/14; A61K 31/44; A61P 29/00; A61K 31/235

(52) U.S. Cl. 424/94.62; 514/643; 514/277; 514/543

(57) ABSTRACT

Disclosed herein are methods for treating mucositis (e.g., Alternaria-activated mucositis) that involves the direct mucoadministration of an active agent that is antifungal and antibacterial in an amount and for a duration effective to treat mucositis.

### RELATED APPLICATIONS

This application is related and claims priority to U.S. Provisional Application No. 60/830,528, filed on Jul. 13, 2006 and U.S. Provisional Application No. 60/900,593, filed on Feb. 9, 2007. The entire contents of both of these applications are incorporated by this reference in their entireties.

### TECHNICAL FIELD

The present invention relates generally to methods for treating mucositis (e.g., non-invasive fungus-induced rhinosinusitis and/or asthma) that include administration of an active agent that is antifungal and antibacterial (e.g., methyl parabens and/or propyl parabens) such that the mucositis is treated (e.g., reduced, eliminated and/or prevented).

### BACKGROUND OF THE INVENTION

Mucositis, the inflammation of mucosal tissue, is a serious medical problem that affects millions of people worldwide. The National Center for Health Statistics describes the increasingly expensive health care burden that chronic rhinosinusitis (CRS) inflicts in the United States. With an estimated 18 to 22 million cases and at least 30 million courses of antibiotics per year, CRS is one of the predominant chronic diseases in the U.S. In 1996, there were 26.7 million visits to physicians, hospital offices, and emergency departments for sinusitis—at a total cost of $5.8 billion. Sinusitis significantly impacts quality of life, even when compared to typical chronic debilitating diseases, such as diabetes and congestive heart failure. CRS presents a challenge to various medical specialties, including infectious diseases, ear, nose, and throat (ENT), allergy, asthma, and clinical immunology. The FDA has not approved any medication for effective use in CRS. Many antibiotic treatments are prescribed without objective evidence of infection. Roughly 600,000 patients per year undergo functional endoscopic sinus surgery, but controlled evidence about the surgical outcomes is lacking. Even with aggressive medical and surgical therapies, many patients have persistent or recurrent disease, leading to frequent courses of antibiotics and multiple surgical interventions.

U.S. Pat. Nos. 6,555,566, 6,291,500 and 6,207,703, by Dr. Jens Ponikau and assigned to the Mayo Foundation For Medical Education And Research, describes and claims methods of treating non-invasive fungus-induced rhinosinusitus, asthma, or intestinal mucositis by directly mucoadministering to at least a portion of the nasal-paranasal anatomy of the subject a formulation including an antifungal in an amount, at a frequency, and for a duration effective to reduce or eliminate the non-invasive fungus-induced rhinosinusitus, asthma, or intestinal mucositis.

U.S. Pat. Nos. 5,785,988, 6,083,525 and 6,344,210, by Charles Fust, are generally directed to compositions for freshening sinus cavities, including a carrier of the ingredients and a masking agent for concealing or eliminating odors that emanate from the sinus cavities. Compositions can include saline solution as a moisturizing base component, a flavoring agent, a preservative, an antiseptic and/or anti-microbial agent (e.g., cetylpyridinium chloride (CPC), triclosan, and benzalkonium chloride), a counter-irritant, and an alcohol. A product called SINOFRESH Essential Nasal Cleansing Formula is sold by SinoFresh Healthcare, Inc. and lists the above-referenced SinoFresh patents. The SINOFRESH package currently states that the formulation includes 0.05% cetylpyridinium chloride antiseptic as the active ingredient, and instructs the user to stop use and ask a doctor if conditions persist for more than 7 days or worsens.

### BRIEF SUMMARY OF THE INVENTION

The present invention is based, at least in part, on the discovery that active agents that are antifungal and antibacterial can be used to treat Alternaria-activated mucositis, e.g., by reduction of Alternaria species in the mucous. Accordingly, in one aspect, the present invention provides methods for treating Alternaria-activated mucositis by directly mucoadministering to a subject in need thereof a composition comprising an active agent that is antifungal and antibacterial such that the Alternaria-activated mucositis is treated. In some embodiments, the Alternaria-activated mucositis is rhinosinusitis, e.g., non-invasive rhinosinusitis. In some embodiments, the Alternaria-activated mucositis is arrested, significantly reduced or eliminated. In some embodiments, the Alternaria-activated mucositis is prevented from re-occurrence.

In another aspect, the present invention provides methods for treating a subject with elevated levels of major basic protein in nasal mucin by directly mucoadministering to the subject a composition comprising an active agent that is antifungal and antibacterial such that the levels of major basic protein in the subject are reduced. In some embodiments, the elevated levels of major basic protein are associated with exposure to an Alternaria species.

In yet another aspect, the present invention provides methods for preventing or arresting fungus-induced inflammation or eosinophil degranulation in a subject by directly mucoadministering to the subject a composition comprising an active agent that is antifungal and antibacterial such that the fungus-induced eosinophil degranulation is prevented. In some embodiments, the inflammation or eosinophil degranulation is associated with exposure to an Alternaria species.

In still another aspect, the present invention provides methods for reducing the load of Alternaria species in a subject by directly mucoadministering to the subject a composition comprising an active agent that is antifungal and antibacterial such that the load of Alternaria species is reduced.

In a further aspect, the present invention provides methods for treating a symptom of Alternaria-activated mucositis by directly mucoadministering to a subject in need thereof a composition comprising an active agent that is antifungal and antibacterial such that at least one symptom of the Alternaria-activated mucositis is treated. Symptoms of Alternaria-activated mucositis include, for example, head pressure, nasal pressure, difficulty breathing, nasal airway obstruction, nasal congestion, nasal discharge, head pain, face pain and decreased sense of smell.

In some embodiments, the Alternaria species is Alternaria alternata. In some embodiments, the active agent includes, for example, antiseptics, methyl and propyl parabens, sodium benzoate, benzyl alcohol, potassium sorbate, sodium metabisulfite, thimerasol, hydrogen peroxide, sodium perborate, polyquad, polyhexamethylene, sodium silver chloride, polyquaternium-1, chlorobutanol.

In some embodiments, the active agent is benzylalkonium chloride. In other embodiments, the active agent is cetylpyridinium chloride. In some embodiments, the active agent includes a methyl paraben, a propyl paraben or combinations thereof. In some embodiments, the active agent is not benzalkonium chloride. In other embodiments, the composition comprises less than 0.005% benzalkonium chloride.

In some embodiments, the active agent comprises a quaternary ammonium salt, e.g., a quaternary ammonium salt of formula (I):

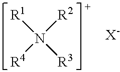

wherein N has a valency of 5;
R¹, R², R³, R⁴ are the same or different and are independently chosen from H, an alkyl group, an alkoxy group, a cycloalkyl group, a heterocycloalkyl group, an aryl group, a heteroaryl group, an acyl group, or a thioacyl group; or two or more of R¹, R², R³ or R⁴ are taken together with the nitrogen to which they are attached, to form a 5-7 membered heterocyclic ring, and
X is an anion.

In some embodiments, X is a halogen. In some embodiments, three of R¹, R², R³ or R⁴ are taken together with the nitrogen to which they are attached to form a 5-7 membered heterocyclic ring. In some embodiments, the compound of formula I is a compound of formula II:

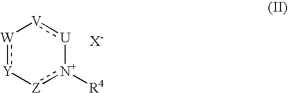

wherein:
U, V, W, Y and Z are each independently selected from the group consisting of CR⁵, CHR⁵, N, NR⁵, O and S;
R⁴ is selected from the group consisting of H, an alkyl group, an alkoxy group, a cycloalkyl group, a heterocycloalkyl group, an aryl group, a heteroaryl group, an acyl group, and a thioacyl group;
R⁵ is selected from the group consisting of H, an alkyl group, an alkoxy group, a cycloalkyl group, a heterocycloalkyl group, an aryl group, a heteroaryl group, an acyl group, and a thioacyl group;
and X is a halogen.

In some embodiments, all three occurrences of

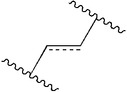

represent double bonds. In some embodiments, U, V, W, Y and Z are each independently CH. In some embodiments, R⁴ is an alkyl group having 6-20 carbon atoms.

In some embodiments, the quaternary ammonium salt has the following structure:

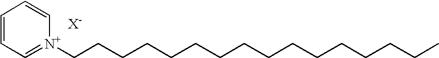

wherein X is an anion, e.g., chlorine.

In some embodiments, methods of the present invention further include co-administering a polysaccharide degrading enzyme, e.g., hyaluronidase. In some embodiments, the hyaluronidase is administered in an amount effective to reduce the viscosity of mucus.

In some embodiments, the amount of the quaternary ammonium salt is between about 0.01% and about 0.5% by weight or volume, e.g., about 0.05% by weight or volume or about 0.02% by weight or volume.

In some embodiments, the formulation further comprises a masking agent.

Compositions of the present invention are administered for an amount of time effective for treatment, e.g., for at least two weeks, e.g., for at least one month. In some embodiments, the formulation administered includes between about 10 μg/ml and about 500 μg/ml active agent.

In some aspects, the present invention is directed to pharmaceutical compositions which include an effective amount of an active agent that is antifungal and antibacterial, a polysaccharide degrading enzyme and a pharmaceutically acceptable carrier. In some embodiments, the polysaccharide degrading enzyme is hyaluronidase.

### DETAILED DESCRIPTION OF THE INVENTION

It has been discovered that certain agents, as described in more detail herein, are surprisingly efficacious against fungal organisms responsible for non-invasive fungal-induced inflammation of the sinus and nasal mucosa, e.g., as demonstrated in the Examples. Accordingly, it is expected that compositions including sufficient amounts of such agents, if administered for a sufficient duration of time (e.g., at least one month), will effectively treat a subject with mucositis, e.g., Alternaria-activated mucositis.

Moreover, the agents of the present invention unexpectedly showed clinical effect (e.g., both antibacterial and antifungal efficacy) at concentrations which are generally only efficacious in non-clinical use. Accordingly, it is expected that administration of these agents in accordance with the methods of the present invention will treat (e.g., retard or eliminate) any bacterial infection present in the mucus of the subject. One advantage of the present invention is that the agents generally are non-toxic or exhibit very low toxicity. Another advantage is that these agents are relatively inexpensive and are readily obtained.

The present invention is based, at least in part, on the discovery that the compositions of the present invention are specifically useful in decreasing the load of Alternaria species. As discussed in more detail herein, immune cells produce cytokines (IL-13 and IL-5) upon exposure to common airborne fungi in patients with mucositis, e.g., chronic rhinosinusitis. Additionally, it was shown that this production of cytokines occurred specifically in connection with the exposure to Alternaria species, and did not occur in healthy control subjects. Moreover, Alternaria species induced a striking degranulation of eosinophils, which degranulation was not induced by other fungal antigens. See, e.g., Inoue Y, et al. “Non-pathogenic, environmental fungi induce activation and degranulation of human eosinophils.” J Immunol 2005; 175:5439-47.

In order to more clearly and concisely describe the subject matter of the claims, the following definitions are intended to provide guidance as to the meaning of specific terms used herein.

Numerous values and ranges are recited in connection with various embodiments of the present invention, e.g., amount of active agent. It is to be understood that all values and ranges which fall between the values and ranges listed are intended to be encompassed by the present invention unless stated otherwise.

It is to be noted that the singular forms “a,” “an,” and “the” as used herein include “at least one” and “one or more” unless stated otherwise. Thus, for example, reference to “a pharmacologically acceptable carrier” includes mixtures of two or more carriers as well as a single carrier, and the like.

As used herein, the term “active agent that is antifungal and antibacterial” refers to any agent which possesses both antifungal and antibacterial properties, including, but not limited to the compounds listed herein. Identification of other equivalent substances is well within the skill of the ordinary practitioner. Single agents with both properties may be advantageous in, e.g., eliminating side effects and/or adverse reactions to combination therapy, ease in manufacturing and/or production, and in lowering the cost of production and treatment.

“Treatment”, or “treating,” as used herein, means the application or administration of a therapeutic agent to a subject who has a disorder, e.g., allergic fungal sinusitis as described herein, with the purpose to cure, heal, alleviate, delay, relieve, alter, remedy, ameliorate, improve or affect the disease or disorder, or symptoms of the disease or disorder. The term “treatment” or “treating” is also used herein in the context of administering agents prophylactically. The term “effective dose” or “effective dosage” is defined as an amount sufficient to achieve or at least partially achieve the desired effect. The term “therapeutically effective dose” is defined as an amount sufficient to cure or at least partially arrest the disease and its complications in a subject already suffering from the disease.

The term “subject,” as used herein, refers to animals such as mammals, including, but not limited to, humans, primates, cows, sheep, goats, horses, pigs, dogs, cats, rabbits, guinea pigs, rats, mice or other bovine, ovine, equine, canine, feline, rodent or murine species.

Fungus is typically present in the air as well as the nasal passages and mucus of subjects. In some subjects, however, an immunologic response results in the symptoms of mucositis. For the purpose of this invention, the term “mucositis” as used herein refers to inflammation of a mucus membrane, and the term “rhinosinusitis” refers to any nasal-paranasal mucositis condition. Additionally, the term “non-invasive fungus-induced rhinosinusitis” includes any nasal-paranasal mucositis condition having a non-invasive fungal etiology. Non-invasive fungus-induced rhinosinusitis can also be referred to as allergic fungal sinusitis (AFS), which is often diagnosed by the presence of inspissated mucus in the nasal-paranasal cavities, which contains clumps or sheets of necrotic eosinophils, Charcot-Leyden crystals, and non-invasive fungal hyphae.

As used herein, the term “fungus-induced eosinophil degranulation” refers to eosinophil degranulation in response to one or more antigens from fungal cells (e.g., from fungal cell extracts or fungal culture supernatants). Eosinophils are the main effectors of antibody-dependent cell-mediated cytotoxicity against multicellular parasites that provoke IgE antibodies. Their role seems to be to engulf and destroy the precipitated antigen-antibody complexes produced in humorally based immune reactions. An elevated eosinophil count usually is seen in allergic reactions, and numerous eosinophils are chemotactically aggregated at sites where antigen-antibody complexes are found. Degranulation is the release of toxic molecules such as eosinophil cationic protein (ECP), eosinophil peroxidase (EPO), and major basic protein (MBP) that are contained within eosinophil granules; this release typically causes damage to or death of cells in the vicinity of the degranulating eosinophils. Degranulation can be assessed by, for example, measuring the release of markers such as ECP, EPO, MBP, or eosinophil derived neurotoxin (EDN). Non-limiting examples of methods for measuring marker levels include protein-based methods such as ELISA assays and western blotting. Alternatively, degranulation can be assessed by visual inspection of eosinophils by microscopy (e.g., using an electron microscope) to detect the presence of empty granules.

As used herein, the term “elevated major basic protein” refers to the situation where levels of major basic protein are higher than those found in healthy individuals (e.g., without Alternaria-activated mucositis). Without wishing to be bound by any particular theory, it is believed that MBP is not measurable or measureable at very low concentrations in healthy individuals (e.g., less than about 0.1 μg/ml). Accordingly, in some embodiments, elevated major basic protein refers to levels of major basic protein of greater than about 0.1 μg/ml. In other embodiments, elevated major basic protein refers to levels of major basic protein of greater than about 0.5 μg/ml. In still other embodiments, elevated major basic protein refers to levels of major basic protein of greater than about 1.0 μg/ml.

The term “chronic” as used herein refers to afflictions present for at least three months. It is to be understood that afflictions that are treated as described herein and become asymptomatic can be classified as chronic. Thus, chronic afflictions can be symptomatic or asymptomatic.

As used herein, the term “mucoadministration” refers to any type of administration that places an administered agent in contact with mucus. Mucoadministration can be, for example, an irrigation of at least a portion of the nasal-paranasal anatomy with a liquid form of the composition. Alternatively, the mucoadministration can involve applying an aerosol form of the composition to at least a portion of the nasal-paranasal anatomy. An active agent of the present invention can be in a solid, liquid, or aerosol form. The term “mucoadministration” can be subdivided into “direct” and “indirect” mucoadministration. The term “direct mucoadministration” as used herein refers to any type of administration that places an administered agent in direct contact with a targeted mucus prior to crossing epithelium. For the purpose of this invention, it is to be understood that injections of an agent into a cavity containing mucus is considered direct mucoadministration if the agent contacts mucus even though an injection means (e.g., needle, tube, or catheter) may be used to cross an epithelium. Thus, using a needle to bypass the tympanic membrane and inject an agent into the middle ear is considered a direct mucoadministration that targets middle ear mucus.

As used herein, the term “Alternaria-activated mucositis” refers to mucositis conditions which are associated with exposure of a subject to at least one Alternaria species.

The term “Alternaria species,” as used herein, refers to at least one species of the genus Alternaria. Alternaria species include, but are not limited to Alternaria alternata, Alternaria arachidis, Alternaria arborescens, Alternaria arbusti, Alternaria blumeae, Alternaria brassicae, Alternaria brassicicola, Alternaria carotiincultae, Alternaria carthami, Alternaria cinerariae, Alternaria citri, Alternaria conjuncta, Alternaria cucumerina, Alternaria dauci, Alternaria dianthicola, Alternaria euphorbiicola, Alternaria gaisen, Alternaria helianthicola, Alternaria infectoria, Alternaria japonica, Alternaria leucanthemi, Alternaria limicola, Alternaria linicola, Alternaria mali, Alternaria molesta, Alternaria padwickii, Alternaria panax, Alternaria petroselini, Alternaria radicina, Alternaria raphani, Alternaria selini, Alternaria senecionis, Alternaria smyrnii, Alternaria solani, Alternaria sonchi, Alternaria tenuissima, Alternaria triticina and Alternaria zinniae. In some embodiments, Alternaria species refers to Alternaria alternata.

As used herein, the term “alkyl group” is intended to mean a straight- or branched-chain monovalent radical of saturated and/or unsaturated carbon atoms and hydrogen atoms, such as methyl (Me), ethyl (Et), propyl, isopropyl, butyl, isobutyl, t-butyl, ethenyl, pentenyl, butenyl, propenyl, ethynyl, butynyl, propynyl, pentynyl, hexynyl, and the like, which may be unsubstituted (i.e., containing only carbon and hydrogen) or substituted by one or more suitable substituents (e.g., one or more halogens, such as F, Cl, Br, or I, with F and Cl being preferred). Preferred alkyl groups are C₁-C₂₀ alkyl groups.

The term “alkoxy group” is intended to mean the radical —ORₐ, where Rₐ is an alkyl group. Exemplary alkoxy groups include methoxy, ethoxy, propoxy, and the like.

A “cycloalkyl group” is intended to mean a non-aromatic monovalent monocyclic, bicyclic, or tricyclic radical containing 3, 4, 5, 6, 7, 8, 9, 10, 11, 12, 13, or 14 carbon ring atoms, each of which may be saturated or unsaturated, and each of which may be unsubstituted or substituted by one or more suitable substituents as defined below, and to which may be fused to one or more heterocycloalkyl groups, aryl groups, or heteroaryl groups, which themselves may be unsubstituted or substituted by one or more substituents.

A “heterocycloalkyl group” is intended to mean a non-aromatic monovalent monocyclic, bicyclic, or tricyclic radical, which is saturated or unsaturated, containing 3, 4, 5, 6, 7, 8, 9, 10, 11, 12, 13, 14, 15, 16, 17, or 18 ring atoms, which includes 1, 2, 3, 4, or 5 heteroatoms such as nitrogen, oxygen, and/or sulfur, where the radical is unsubstituted or substituted by one or more suitable substituents as defined below, and to which may be fused one or more cycloalkyl groups, aryl groups, or heteroaryl groups, which themselves may be unsubstituted or substituted by one or more suitable substituents.

An “aryl group” is intended to mean an aromatic monovalent monocyclic, bicyclic, or tricyclic radical containing 6, 10, 14, or 18 carbon ring atoms, which may be unsubstituted or substituted by one or more suitable substituents as defined below, and to which may be fused one or more cycloalkyl groups, heterocycloalkyl groups, or heteroaryl groups, which themselves may be unsubstituted or substituted by one or more suitable substituents.

A “heteroaryl group” is intended to mean an aromatic monovalent monocyclic, bicyclic, or tricyclic radical containing 4, 5, 6, 7, 8, 9, 10, 11, 12, 13, 14, 15, 16, 17, or 18 ring atoms, including 1, 2, 3, 4, or 5 heteroatoms such as nitrogen, oxygen, and/or sulfur, which may be unsubstituted or substituted by one or more suitable substituents as defined below, and to which may be fused one or more cycloalkyl groups, heterocycloalkyl groups, or aryl groups, which themselves may be unsubstituted or substituted by one or more suitable substituents.

An “acyl group” is intended to mean a —C(O)—R radical, where R is a substituent.

A “thioacyl group” is intended to mean a—C(S)—R radical, where R is a substituent.

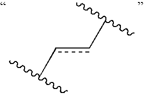

As used herein, a dotted line refers to an optional bond. That is, refers to a moiety which may be a single bond or a double bond.

As used herein, the term “substituent” is meant to include all permissible substituents of organic compounds. In a broad aspect, the permissible substituents include acyclic and cyclic, branched and unbranched, carbocyclic and heterocyclic, aromatic and nonaromatic substituents of organic compounds. In some embodiments, “substituent” refers to moieties including halogen, haloalkyl, nitro, cyano, alkyl, alkenyl, alkynyl, cycloalkyl, alkoxy, heterocyclyl, aralkyl, aryl and heteroaryl.

It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. It is also understood that “substitution” or “substituted with” includes one or more substituents.

In some embodiments, the compositions and methods of the present invention do not include those listed in U.S. Patent Application Publication No. US 2005/0084454, U.S. Pat. Nos. 5,785,988, 6,083,525 and/or 6,344,210. The contents of these references are incorporated in their entireties by this reference. In one embodiment of the invention, the active agent is not any of the compounds and compound classes disclosed in U.S. Pat. Nos. 6,555,566, 6,291,500 and 6,207,703. The contents of these references are incorporated in their entireties by this reference.

In some aspects, the present invention is generally directed to methods for treating Alternaria-activated mucositis in a subject that include the direct mucoadministration of an active agent that is antifungal and antibacterial in an amount effective to treat the Alternaria-activated mucositis.

Without wishing to be bound by any particular theory, it is believed that fungal antigens trigger lymphocytes to produce cytokines (e.g., IL-13 and IL-5), which are responsible for a portion of eosinophil activity. Eosinophils are largely involved in the body's defense against larger organisms. In the mucus of subjects with mucositis, e.g., CRS, activation of eosinophils eventually results in degranulation and release of cytotoxic proteins, such as eosinophilic major basic protein (MBP). Although MBP is toxic to the fungi, it also damages the nasal epithelium, which eventually results in inflammation and airway remodeling as well as secondary infections due to the vulnerability of damaged tissue to bacterial and viral invasion. It has recently been shown that peripheral blood mononuclear cells (PBMC) about 90% of tested CRS patients, but not those from normal tested individuals, produced both IL-5 and IL-13 when exposed to Alternaria, Aspergillus, or Cladosporium. Moreover, in response to Alternaria, PBMC from CRS patients produced about 5-times more IFN-γ than PBMC from normal individuals. Overall, CRS patients tend to exhibit exaggerated humoral and cellular responses, both Th1 and Th2 types, to common airborne fungi, particularly Alternaria. It has also been shown that Alternaria cell extracts and culture supernatants are particularly useful in the degranulation of eosinophils, possibly due to the production of PAR-activating enzymes during germination and growth. See, e.g., U.S. Patent Application Publication No. 20070154987, the entire contents of which are hereby incorporated by this reference. Accordingly, it is expected that reduction in Alternaria loads in the mucosa will lead to prevention, slowing or arresting of fungus-induced eosinophil degranulation. Moreover, it is believed that the prevention, slowing or arresting of fungus-induced eosinophil degranulation will in turn lead to lower levels of cytotoxic proteins.

Accordingly, in other aspects, the present invention is generally directed to methods for treating elevated levels of major basic protein in a subject that include the direct mucoadministration of an active agent that is antifungal and antibacterial in an amount effective to reduce the levels of major basic protein in the subject. In yet other aspects, the present invention is generally directed to methods for preventing or arresting fungus-induced eosinophil degranulation in a subject that include the direct mucoadministration of an active agent that is antifungal and antibacterial in an amount effective to prevent or arrest the fungus-induced eosinophil degranulation in the subject.

In another aspect, the invention features methods for treating chronic asthma by directly mucoadministering a composition comprising an active agent that is antifungal and antibacterial. It has also been shown that asthma and chronic rhinosinusitis coexist clinically in >50% of patients with chronic rhinosinusitis. Accordingly, it is to be understood that any combination of the above-indicated disorders or symptoms of the above-indicated disorders may be treated by the methods of the present invention (e.g., asthma and non-invasive fungus-induced rhinosinusitis).

In yet another aspect, the present invention provides methods for treating a symptom of Alternaria-activated mucositis by directly mucoadministering to a subject in need thereof a composition comprising an active agent that is antifungal and antibacterial such that at least one symptom of the Alternaria-activated mucositis is treated. Such symptoms include, but are not limited to, head pressure, nasal pressure, difficulty breathing, nasal airway obstruction, nasal congestion, nasal discharge, head pain, face pain and decreased sense of smell.

In some embodiments, the methods of the present invention include co-administration of the composition with a polysaccharide degrading enzyme. As used herein, the term “polysaccharide degrading enzyme” refers to an enzyme that cleaves glycosidic bonds. Without wishing to be bound by any particular theory, it is believed that such an enzyme would cleave the glycosidic bonds of polysaccharides present in mucus and, thereby aid in breaking up thick secretions, e.g., by reducing the viscosity of mucus. Examples of a polysaccharide degrading enzyme include, but are not limited to, β-glucosidase, pullulanase, neuraminidase and hyaluronidase. In a particular embodiment, the polysaccharide degrading enzyme is hyaluronidase.

Accordingly, in another aspect, the present invention provides pharmaceutical compositions which include an effective amount of an active agent that is antifungal and antibacterial, a polysaccharide degrading enzyme and a pharmaceutically acceptable carrier. The polysaccharide degrading enzyme can be, e.g., any polysaccharide degrading enzyme listed above. In one embodiment, the polysaccharide degrading enzyme comprises hyaluronidase.

In some embodiments, the methods of the present invention treat one or more disorders by arresting, significantly alleviating or curing the disorder being treated. In other embodiments, the methods are directed toward preventing re-occurrence of the disorders treated using the present invention.

In some embodiments, the active agent of the composition used in the present invention includes at least one agent selected from the group consisting of: antiseptics, methyl and propyl parabens, sodium benzoate, benzyl alcohol, potassium sorbate, sodium metabisulfite, thimerasol, hydrogen peroxide, sodium perborate, polyquad, polyhexamethylene, sodium silver chloride, polyquaternium-1, chlorobutanol. In other embodiments of the present invention, the active agent is benzylalkonium chloride, a mixture of alkylbenzyl dimethylammonium chlorides of various alkyl chain lengths. In yet another embodiment of the invention, the active agent involves an alkyl paraben, e.g., a methyl paraben, a propyl paraben or combinations of both.

Examples of antiseptics include, but are not limited to aliphatic alcohols such as ethanol, n-propanol and isopropanol; halogenated aliphatic alcohols such as chlorobutanol and 2-bromo-2-nitro-propanol-1,3-diol (to be abbreviated as bronopol hereinafter); aromatic alcohols such as 2,4-dichlorobenzyl alcohol, 2-phenoxyethanol, phenoxyisopropanol, phenylethyl alcohol and 3-(4-chlorophenoxy)-1,2-propane diol; aldehydes such as 5-bromo-5-nitro-1,3-dioxane, formaldehyde, paraformaldehyde and glutaraldehyde; gradually-liberating agents capable of forming an aldehyde under acidic condition, such as hexamethylenetetramine, monomethylol dimethyl hydantoin and dimethylol methyl hydantoin; amides such as chloroacetoamide; ureas such as N,N′-methylene-bis(N′-(1-(hydroxymethyl)-2,5-dioxo-4-imidazolidinyl)urea and N-(hydroxymethyl)-N-(1,3-dihydroxymethyl-2,5-dioxo-4-imidazolidinyl)-N′-(hydroxymethyl)urea; inorganic sulfites, bisulfites and pyrosulfites such as sodium sulfite, potassium sulfite, sodium bisulfite, potassium bisulfite, sodium pyrosulfite and potassium pyrosulfite; inorganic acids such as boric acid; organic acid compounds such as formic acid, propionic acid, 10-undecylenic acid, sorbic acid, benzoic acid, salicylic acid and 2-acetyl-5-hydroxy-3-oxo-4-hexanoic acid δ lactone; antibiotics such as 2,6-diacetyl-7,9-dihydroxy-8,9b-dimethyl-1,3-(2H,9bH)-dibenzoflandion; p-hydroxy benzoate compounds such as methyl p-hydroxy benzoate, ethyl p-hydroxy benzoate, n-propyl p-hydroxy benzoate, n-isopropyl p-hydroxy benzoate, n-butyl p-hydroxy benzoate, n-isobutyl p-hydroxy benzoate, t-butyl p-hydroxy benzoate and benzyl p-hydroxy benzoate; halogenated phenol compounds such as 4-chloro-3-methyl phenol, 4-chloro-3,5-xylenol, 3,4,5,6-tetrabromo-O-cresol, 2,4-dichloro-3,5-xylenol, 2-benzyl-4-chloro-phenol, 2,2′-methylene-bis-(4-chlorophenol), 3,3′-dibromo-5,5′-dichloro-2,2′-dihydroxy-diphenylmethane and 2,2′-methylene-bis(3,4,6-trichlorophenol); phenol compounds such as 4-chloro-5-methyl-2-(1-methylethyl)phenol, 1-methyl-2-hydroxy-4-isopropyl benzene, 2-phenyl phenol and 4-isopropyl-3-methyl-phenol; diphenyl ether compounds such as 2,4,4′-trichloro-2′-hydroxydiphenyl ether; carbonilide compounds such as 3,4,40-trichlorocarbanilide and 4,4′-dichloro-3-(3-fluoromethyl)carbanilide; benzamidine compounds such as 4,4′-diamidino-α, ω-diphenoxypropane isethionate, 4,4′-(trimethylenedioxane)-bis-(3-bromobenzamidine diisethionate (hereinafter referred to as dibromopropamidine) and 1,6-di(4-amidinophenoxy)-n-hexane(hexamidine isethionate); cyclic thiohydroxamic acids and salts thereof such as pyridine-1-oxide-2-thiol-sodium salts, zinc bis-(2-pyridinethiol-1-oxide)bis-(2-pyridylthio)zinc-1,1′-dioxide(zinc pyrithione); N-acetal compounds such as 5-amino-1,3-bis(2-ethylhexyl)-5-methylhexahydropyrimidine(hexsetidine) and tris-hydroxyethylhexahydrotriazine; phthalimide derivatives such as N-(trichloromethylthio)-4-cyclohexane-1,2-dicarboxylmide(captane); o-acetal compounds such as 6acetoxy-2,4-dimethyl-m-dioxane(dimethoxane); oxazolidine compounds such as 4,4-dimethyl-1,3-oxazolidine(oxazine A); quinoline compounds such as 8-hydroxyquinoline; cationic substances such as bis(p-chlorophenyldiguanide)hexane and polyhexamethylenebiguanide hydrochloride; quarterly salt compounds such as alkyltrimethylammonium bromide, N-dodecyl-N,N-dimethylbenzyl ammonium, and N,N-dimethyl-N-(2-(2-(4-(1,1,3,4-tetramethylbutyl)phenoxy)ethoxy)-ethyl)benzene methane ammonium chloride; organic mercury compounds such as ethyl mercury thiosalicylate and phenyl acetate mercury; iodine compounds such as sodium iodate; glyceryl monolaurates; pyridone derivatives such as 1-hydroxy-4-methyl-6-(2,4,4-trimethylpentyl)-2-(1H)pyridone ethanol amine salt; and the like. It is desirable that an antiseptic which does not agglomerate emulsion particles remarkably or does not impair occlusion of dentinal tubules is selected from among these antiseptics.

In a further embodiment of the present invention, the active agent involves a quaternary ammonium salt. In yet a further embodiment, the active agent involves at least quaternary ammonium salt of formula I:

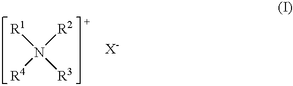

wherein N has a valency of 5;

R¹, R², R³, and R⁴ are the same or different and are independently selected from the group consisting of H, an alkyl group, an alkoxy group, a cycloalkyl group, a heterocycloalkyl group, an aryl group, a heteroaryl group, an acyl group, and a thioacyl group; or two or more of R¹, R², R³ or R⁴ are taken together with the nitrogen to which they are attached, to form a 5-7 membered heterocyclic ring, and

X is an anion, preferably a halogen, e.g., Cl, Br, I, or F.

In one embodiment, two of R¹, R², R³ or R⁴ are taken together to form a 5-7 membered heterocyclic ring.

In another embodiment, three of R¹, R², R³ or R⁴ are taken together to form a 5-7 membered heterocyclic ring.

In a further embodiment, the compound of the present invention is a compound of formula II:

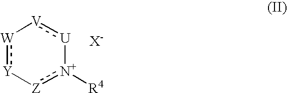

wherein:

U, V, W, Y and Z are each independently selected from the group consisting of CR⁵, CHR⁵, N, NR⁵, O and S;

R⁴ is selected from the group consisting of H, an alkyl group, an alkoxy group, a cycloalkyl group, a heterocycloalkyl group, an aryl group, a heteroaryl group, an acyl group, and a thioacyl group;

R⁵ is selected from the group consisting of H, an alkyl group, an alkoxy group, a cycloalkyl group, a heterocycloalkyl group, an aryl group, a heteroaryl group, an acyl group, and a thioacyl group;

and X is a halogen.

In some embodiments, one or more

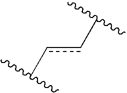

represents a double bond. In some embodiments, all three of

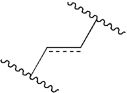

represent double bonds.

In some embodiments, U, V, W, Y and Z are each independently CH.

In one embodiment, R⁴ is an alkyl group having 6-20 carbon atoms. In a further embodiment, R⁴ is an alkyl group having 8-18 carbon atoms. In yet another embodiment, R⁴ is an alkyl group having 10-16 carbon atoms.

In one embodiment, X is Cl. In one embodiment, X is Br.

In another further embodiment, the quaternary ammonium salt is a cetylpyridinium moeity, i.e.,

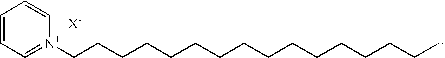

In some embodiments, the quaternary ammonium salt is cetylpyridinium chloride. In some embodiments, the quaternary ammonium salt is cetylpyridinium bromide.

In the present invention, the amount of the quaternary ammonium salt used in the composition is between about 0.001% and about 1.0% by weight or volume. In some embodiments, the amount of the quaternary salt is about 0.002% by weight or volume. In some embodiments, the amount of the quaternary salt is about 0.005% by weight or volume. In some embodiments, the amount of the quaternary salt is about 0.01% by weight or volume. In some embodiments, the amount of the quaternary salt is about 0.02% by weight or volume. In some embodiments, the amount of the quaternary salt is about 0.03% by weight or volume. In some embodiments, the amount of the quaternary salt is about 0.05% by weight or volume. In some embodiments, the amount of the quaternary salt is about 0.05% by weight or volume. In some embodiments, the amount of the quaternary salt is about 0.075% by weight or volume. In other embodiments, the amount of quaternary salt is about 0.1% by weight or volume. In some embodiments, the amount of the quaternary salt is about 0.15% by weight or volume. In yet other embodiments, the amount of quaternary salt is about 0.25% by weight or volume. In yet another embodiment, the amount of quaternary salt is about 0.4% by weight or volume. In yet another embodiment, the amount of quaternary salt is about 0.5% by weight or volume. It is to be understood that all values and ranges which fall between the values and ranges listed above are intended to be encompassed by the present invention.

In some embodiments of the present invention, the formulation administered involves between about 10 μg/ml and about 500 μg/ml active agent. In some embodiments, the formulation administered involves about 25 μg/ml active agent. In other embodiments, the formulation administered involves about 50 μg/ml active agent. In yet other embodiments, the formulation administered involves about 100 μg/ml active agent. In another embodiment, the formulation administered involves about 250 μg/ml active agent. In yet another embodiment, the formulation administered involves about 400 μg/ml active agent. It is to be understood that all values and ranges which fall between the values and ranges listed above are intended to be encompassed by the present invention.

Another embodiment of the present invention involves administering the formulation for a duration and frequency sufficient to treat mucositis. In some embodiments, the formulation will be administered for at least two weeks. In some embodiments, the formulation will be administered for at least three weeks. In other embodiments, the formulation will be administered for at least one month. In some embodiments, the formulation will be administered for at least three months. In some embodiments, the formulation will be administered for at least six months. In some embodiments, the formulation will be administered for at least nine months. In yet another embodiment, the formulation will be administered for at least one year.

In some embodiments of the present invention, the formulation further involves a masking agent. In some embodiments, the masking agent is any agent known to mask the taste or smell of another agent, e.g., the active agent. The masking agent can be, but is not limited to a flavoring agent such as, for example, peppermint oil, or fruit essence.

In one embodiment, the active agent is not cetylpyridinium chloride. In one embodiment, the active agent is not a quaternary ammonium salt. In one embodiment, the active agent is not benzylalkonium chloride.

In some embodiments, the present invention provides methods of treatment using compositions which include benzalkonium in an amount less than an amount effective for treating non-invasive fungus-induced rhinosinusitis. In some embodiments, an amount effective for treating non-invasive fungus-induced rhinosinusitis is about 0.025%. In some embodiments, an amount effective for treating non-invasive fungus-induced rhinosinusitis is about 0.020%. In some embodiments, an amount effective for treating non-invasive fungus-induced rhinosinusitis is about 0.015%. In some embodiments, an amount effective for treating non-invasive fungus-induced rhinosinusitis is about 0.010%. In some embodiments, an amount effective for treating non-invasive fungus-induced rhinosinusitis is about 0.005%. In some embodiments, an amount effective for treating non-invasive fungus-induced rhinosinusitis is about 0.0025%. In some embodiments, an amount effective for treating non-invasive fungus-induced rhinosinusitis is about 0.001%.

Practice of the invention will still more fully be understood from the following examples, which are presented herein for illustration purposes only and should not be construed as limiting the invention in any way.

### EXEMPLIFICATION

### Example 1

### Inhibition of Alternaria alternata

SINOFRESH Essential Nasal Cleansing Formula, which included cetylpyridinium chloride (CPC) at 0.02% and benzalkonium chloride (BKC) at 0.005%, was tested for its ability to inhibit Alternaria alternata, a fungus typically associated with non-invasive fungus-induced rhinosinusitis. Minimum Inhibitory Concentration (MIC), Antifungal Efficacy, and Selective Agar Streak were all determined, and met the pre-defined acceptance criteria that the negative control test tube had to remain free of growth throughout the study, while the positive control tube had to retain growth throughout the study. No deviations were noted during the study.

Study Results

Minimum Inhibitory Concentration

Minimum Inhibitory Concentration (MIC) measures the amount of growth contained in a sample at the macroscopic level. A rating of zero indicates that growth equal to the negative control was observed, while a four indicates that growth equal to the positive control was observed. Tubes containing the sample was tested with (indicated by the word “spike”) and without 1×10⁵ Alternaria alternata for the amount of growth present.

Based on the results as shown in Table I, there was no macroscopic growth present in the unspiked tubes. In the spiked tubes, all concentrations of CPC 0.02% BKC 0.005% were able to inhibit most macroscopic mold growth. Refer to the antifungal efficacy results below for actual growth present in the 100% sample tube.

TABLE I
MIC Results

| | | CPC 0.02% |
|---|---|---|
| | CPC 0.02% | BKC 0.005% |
| Concentration | BKC 0.005% | Spike |
| 100% | 0 | 1 |
| 75% | 0 | 1 |
| 50% | 0 | 1 |
| 25% | 0 | 1 |
| 12.5% | 0 | 1 |

Positive control tube = 4, Negative control tube = 0

Antifungal Efficacy

A 100% sample of 0.02% cetylpyridinium chloride was tested for antifungal efficacy. At 1, 4, 24, and 48 hours of incubation, the sample was diluted and plated. On days 2, 3, and 5, the plate was read. The plate did not exhibit any mold growth.

Selective Afar Streak

The selective agar streak test was performed to monitor the type(s) of bacterial contamination present in the MIC tubes. After 48 hours of incubation, all MIC tubes were streaked onto four different selective agars: Pseudocel, Mannitol Salt, MacConkey, and Brilliant Green.

No bacterial growth was present in any tubes with CPC 0.02% BKC 0.005%, the MIC positive control plates, the MIC negative control plates, or the media negative control plates. Growth was present on all plates containing positive control organisms. No growth was present on any Pseudocel or Mannitol Salt agar plates, indicating that bacteria of the genera Pseudomonas and Staphylococcus were not present in any MIC tube. Bacterial growth was present on all MacConkey and Brilliant Green agar plates for all.

The same inhibition studies were run for Amphotericin B (0.1 mg/ml) under the same conditions. Data from the Amphotericin B studies are not included herein because it was established that the Amphotericin B preparation was not fresh, and it is believed that the preparation may have degraded under the storage conditions used, and thus provided inconsistent results. Positive results for Amphotericin B have been previously established, and may be found, for example, in U.S. Pat. No. 6,555,566 or in Ponikau J. U., et al. “Intranasal antifungal treatment in 51 patients with chronic rhinosinusitis.” J Allergy Clin Immunol. 2002 December; 110(6):862-6.

### Example 2

### Inhibition of Alternaria alternata by Parabens compared to Amphotericin-B

Amphotericin-B and Parabens (methylparaben and propylparaben) were tested for their ability to inhibit the fungus Alternaria alternata. Samples were stored at 2-8° C. prior to use.

Study Design

Prepared an inoculating culture of Alternaria alternata by placing an ATCC pellet in 5 mL phosphate buffer, after dissolution spread the broth and dissolved pellet over the surface of a PDA plate using a FIG. 8 motion. The inoculum plate was incubated for five days at 20-25° C.

After the five day incubation period, 3 ml of modified phosphate buffer was used to wet the surface growth of the PDA plate. The surface of the culture was washed with the buffer using a sterile spreader stick. After washing, a 10 mL pipette was used to remove as much of the wash as is possible without picking up any agar. The wash was pipetted into a sterile culture tube.

Serial dilutions of the wash modified phosphate buffer were performed using modified phosphate buffer to determine the dilution that yields a mold population of 10⁶ per mL. The dilutions were done by pipetting 1 mL of each dilution of the wash into molten potato dextrose agar plates pH 3.5 in quadruplicate, incubating at 20-25° C. for five days, assuring that there are not mixed cultures, and then counting the colonies on the plates. The correct dilution yielded counts of 30-300 CFU's. To maintain the correct dilution for inoculation, all dilutions were refrigerated at 2-8° C. until needed. At the end of five days, the dilution selected for further use had a CFU/mL count of 106. A 0.1 mL aliquot of this dilution was used to inoculate the inhibition culture tubes.

Twelve sterile test tubes were prepared with 9 mL Modified Synthetic Medium RPMI-1640 broth that has been tempered in a 45-50° C. water bath and labeled as follows: Amphotericin B neg. control, Ampho-B 100 spike, Ampho-B 2 spike, Ampho-B 1 spike, Ampho-B 0.5 spike, Paraben neg. control, Paraben 400/100 spike, Paraben 300/75 spike, Paraben 200/50 spike, Paraben 100/25 spike, Pos. control, Neg control.

An Amphotericin-B suspension was prepared by placing in suspension 10 mg of Amphotericin-B in 10 mL in modified phosphate buffer. The sample was mixed by vortex for 30 seconds.

A paraben stock solution was prepared by dissolving 80 mg methyl paraben and 20 mg propyl paraben in 20 mL of modified Phosphate buffer heated to 80-90° C. with constant agitation. The stock solution was filtered using a sterile filter in a biological safety cabinet prior to use.

Control samples, Amphotericin B and Parabens dilutions were all prepared from the stock solutions. To all tube labeled “spike” and positive control, 0.1 mL of the inoculated buffer was added and determined to yield 10 to each tube. The tubes were incubated at 30-35° for 48 hours. The growth in each tube was recorded visually, on a 0-4 scale: 0: no growth observed, 1: slight growth or ˜25% of the growth control, 2: prominent growth or ˜50% of the growth control, 3: slight reduction in growth or ˜75% of the growth control, and 4: no reduction in growth or 100% of the growth control. The MIC is the lowest concentration that prevents any visible growth (0 on this scale).

Study Results

Results from the inhibition of Alternaria alternata for Amphotericin B and Parabens are shown in Table II.

TABLE II

| Sample | Rating |
|---|---|
| Positive Control | 4 |
| Negative Control | 0 |
| Negative Control (Parabens) | 0 |
| Negative Control (Amphotericin B) | 0 |
| Amphotericin B 0.5 μg/mL | 2 |
| Amphotericin B 1.0 μg/mL | 2 |
| Amphotericin B 2.0 μg/mL | 1 |
| Amphotericin B 100 μg/mL | 1 |
| Parabens 50/12.5 μg/mL | 4 |
| Parabens 100/25 μg/mL | 4 |
| Parabens 200/50 μg/mL | 2 |
| Parabens 300/75 μg/mL | 2 |
| Parabens400/100 μg/mL | 0-1* |

0 = no growth, 4 = 100% growth as compared to positive control
*= visible growth, but less than 25% of positive control

Deviations

The deviations noted were: Sabouraud dextrose agar (SDA) was used in place of potato dextrose agar (PDA) for dilution plates on the 11^{th} and 17^{th} day of the study; the Amphotericin-B and Parabens solutions used were not filtered; and the second and third sets of dilution plates was incubated for four days instead of five as adequate growth was noted to perform plate count.

### Example 3

### Inhibition of Alternaria by Cetyl Pyridinium Chloride, Benzalkonium Chloride or a Combination of Both

Cetyl pyridinium chloride (CPC) and benzalkonium chloride (BKC) were both independently tested to determine their efficacy of inhibiting Alternaria alternata, a fungus typically associated with non-invasive fungus-induced rhinosinusitis. An in vitro test of the efficacy of BKC and CPC in combination was also completed against Alternaria alternata.

Five concentrations of BKC and CPC were independently tested against Alternaria alternata in a cylinder Agar Plate Assay. The CPC concentrations were 0.005%, 0.01%, 0.05%, 0.25% and 1.0%, whereas the BKC concentrations were 0.00025%, 0.001%, 0.005%, 0.025%, and 0.1%. These concentrations were chosen to include the concentrations in the SINOFRESH product as well as two concentrations above and two concentrations below the SINOFRESH product. BKC at the concentration 0.005% had little to no effect on the growth of Alternaria alternata. BKC at concentrations of about 0.025% or above had some effect on the growth of Alternaria alternata. The most observable effect on the growth of Alternaria alternata was caused by CPC, independently, at the 5 different concentrations.

Five concentrations of a combination of BKC and CPC were independently tested against Alternaria alternata in a cylinder Agar Plate Assay. The concentrations were as follows:

| Sample | CPC % | BKC % |
|---|---|---|
| CPC & BKC 1 | 0.05 | 0.005 |
| CPC & BKC 2 | 0.005 | 0.0005 |
| CPC & BKC 3 | 0.0005 | 0.00005 |
| CPC & BKC 4 | 1/10th of Sample 3 | 1/10th of Sample 3 |
| CPC & BKC 5 | 1/100th of Sample 3 | 1/100th of Sample 3 |

Five concentrations of the SINOFRESH product were also subjected to the same Agar Plate Assay, with the same concentrations of BKC and CPC. In both instances, samples with a concentration of CPC greater than about 0.005% were able to provide an observable effect on the growth of Alternaria alternata

It appears that the anti-Alternaria alternata activity is due mainly to the CPC. As can be seen from the figures, higher concentrations of BKC were needed for activity against Alternaria alternata, and the BKC was essentially inactive at the concentrations in the SINOFRESH Product.

### Equivalents/Other Embodiments

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes which come within the meaning and range of equivalency of the claims are therefore intended to be embraced herein.

What is claimed is:

1. A method for treating Alternaria-activated mucositis, comprising directly mucoadministering to a subject in need thereof a composition comprising an active agent that is antifungal and antibacterial such that the Alternaria-activated mucositis is treated.

2. The method of claim 1, wherein the Alternaria-activated mucositis is rhinosinusitis.

3. The method according to any of the preceding claims, wherein the Alternaria-activated mucositis is non-invasive rhinosinusitis.

4. The method according to any of the preceding claims, wherein the Alternaria-activated mucositis is arrested, significantly reduced or eliminated.

5. The method according to any of the preceding claims, wherein the Alternaria-activated mucositis is prevented from re-occurrence.

6. A method for treating a subject with elevated levels of major basic protein in nasal mucin, comprising directly mucoadministering to the subject a composition comprising an active agent that is antifungal and antibacterial such that the levels of major basic protein in the subject are reduced.

7. The method according to claim 6, wherein the elevated levels of major basic protein are associated with exposure to an Alternaria species.

8. A method for preventing or arresting fungus-induced inflammation or eosinophil degranulation in a subject, comprising directly mucoadministering to the subject a composition comprising an active agent that is antifungal and antibacterial such that the fungus-induced eosinophil degranulation is prevented.

9. The method according to claim 8, wherein the inflammation or eosinophil degranulation is associated with exposure to an Alternaria species.

10. A method for reducing the load of Alternaria species in a subject, comprising directly mucoadministering to the subject a composition comprising an active agent that is antifungal and antibacterial such that the load of Alternaria species is reduced.

11. A method for treating a symptom of Alternaria-activated mucositis, comprising directly mucoadministering to a subject in need thereof a composition comprising an active agent that is antifungal and antibacterial such that at least one symptom of the Alternaria-activated mucositis is treated.

12. The method according to claim 11, wherein symptoms of Alternaria-activated mucositis comprise head pressure, nasal pressure, difficulty breathing, nasal airway obstruction, nasal congestion, nasal discharge, head pain, face pain and decreased sense of smell.

13. The method according to any of claims 1-5, 7 or 9-12, wherein the Alternaria species is Alternaria alternata.

14. The method according to any of the preceding claims, wherein the active agent comprises at least one agent selected from the group consisting of: antiseptics, methyl and propyl parabens, sodium benzoate, benzyl alcohol, potassium sorbate, sodium metabisulfite, thimerasol, hydrogen peroxide, sodium perborate, polyquad, polyhexamethylene, sodium silver chloride, polyquaternium-1, chlorobutanol.

15. The method according to any one of claims 1-14, wherein the active agent is benzylalkonium chloride.

16. The method according to any one of claims 1-14, wherein the active agent is cetylpyridinium chloride.

17. The method according to any one of claims 1-14, wherein the active agent comprises a methyl paraben, a propyl paraben or combinations of both.

18. The method according to any one of claims 1-14, wherein the active agent comprises a quaternary ammonium salt.

19. The method of any of claims 1-14, wherein the active agent comprises at least one quaternary ammonium salt of formula (I): 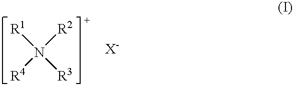
wherein N has a valency of 5;
R¹, R², R³, R⁴ are the same or different and are independently chosen from H, an alkyl group, an alkoxy group, a cycloalkyl group, a heterocycloalkyl group, an aryl group, a heteroaryl group, an acyl group, or a thioacyl group; or two or more of R¹, R², R³ or R⁴ are taken together with the nitrogen to which they are attached, to form a 5-7 membered heterocyclic ring, and
X is an anion.

20. The method of claim 19, wherein X is a halogen.

21. The method of claim 19 or claim 20, wherein three of R¹, R², R³ or R⁴ are taken together with the nitrogen to which they are attached to form a 5-7 membered heterocyclic ring.

22. The method of any of claims claim 19-21, wherein the compound of formula I is a compound of formula II: 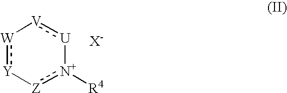
wherein:
U, V, W, Y and Z are each independently selected from the group consisting of CR⁵, CHR⁵, N, NR⁵, O and S;
R⁴ is selected from the group consisting of H, an alkyl group, an alkoxy group, a cycloalkyl group, a heterocycloalkyl group, an aryl group, a heteroaryl group, an acyl group, and a thioacyl group;
R⁵ is selected from the group consisting of H, an alkyl group, an alkoxy group, a cycloalkyl group, a heterocycloalkyl group, an aryl group, a heteroaryl group, an acyl group, and a thioacyl group;
and X is a halogen.

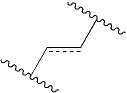

23. The method of any of claims 19-22, wherein all three occurrences of represent double bonds.

24. The method of any of claims 19-23, wherein U, V, W, Y and Z are each independently CH.

25. The method of any of claims 19-24, wherein R⁴ is an alkyl group having 6-20 carbon atoms.

26. The method of any of claims 19-25, wherein the quaternary ammonium salt has the following structure: 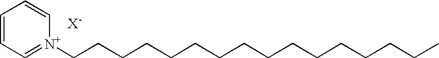
wherein X is an anion.

27. The method according to any of claims 19-26, wherein X is chlorine.

28. The method according to any of the preceding claims, further comprising co-administering a polysaccharide degrading enzyme.

29. The method according to any of the preceding claims, further comprising co-administering hyaluronidase.

30. The method of claim 29, wherein the hyaluronidase is administered in an amount effective to reduce the viscosity of mucus.

31. The method of any of claims 18-30, wherein the amount of the quaternary ammonium salt is between about 0.01% and about 0.5% by weight or volume.

32. The method of any of claims 18-30, wherein the amount of the quaternary ammonium salt is about 0.05% by weight or volume.

33. The method of any of claims 18-30, wherein the amount of the quaternary ammonium salt is about 0.02% by weight or volume.

34. The method of any of the preceding claims, comprising administering the formulation for at least two weeks.

35. The method of any of the preceding claims, comprising administering the formulation for at least one month.

36. The method of any of the preceding claims, wherein the formulation further comprises a masking agent.

37. The method of any of the preceding claims, wherein the formulation administered comprises between about 10 μg/ml and about 500 μg/ml active agent.

38. The method of any of the preceding claims, wherein said active agent is not benzalkonium chloride.

39. The method of any of the preceding claims, wherein the composition comprises less than about 0.005% benzalkonium chloride.

40. A pharmaceutical composition comprising an effective amount of an active agent that is antifungal and antibacterial, a polysaccharide degrading enzyme and a pharmaceutically acceptable carrier.

41. The pharmaceutical composition of claim 40, wherein the polysaccharide degrading enzyme is hyaluronidase.
